(19) United States
(12) Patent Application Publication
(10) Pub. No.: US 2008/0027513 A1
(43) Pub. Date: Jan. 31, 2008

(54) Systems And Methods For Using A Butterfly Coil To Communicate With Or Transfer Power To An Implantable Medical Device

(75) Inventor: Rafael Carbunaru, Studio City, CA (US)

Correspondence Address: Wong, Cabello, Lutsch, Rutherfor & Brucculer L.L.P, 20333 SH 249, Suite 600, Houston, TX, 77070, US

(73) Assignee: ADVANCED BIONICS CORPORATION, Valencia, CA (US)

(21) Appl. No.: 11/630,708

(22) PCT Filed: Jul. 8, 2005
(86) PCT No.: PCT/US05/24303

§ 371 Date: Dec. 20, 2006

Related U.S. Application Data
(60) Provisional application No. 60/586,864, filed on Jul. 9, 2004.

(51) Int. Cl. A61N 1/378

(52) U.S. Cl. 607/60; 607/2; 607/65

(57) ABSTRACT

Systems for communicating with or transferring power to an implantable medical device include a primary coil configured to emit a magnetic field and a secondary coil in the implantable medical device configured to receive the magnetic field. The primary coil includes at least one butterfly coil. Methods of communicating with or transferring power to an implantable medical device include emitting a magnetic field with a primary coil and receiving the magnetic field with a secondary coil. The primary coil includes at least one butterfly coil.

### RELATED APPLICATIONS

The present application claims the benefit under 35 U.S.C. §119(e) of U.S. Provisional Patent Application No. 60/586,864, filed Jul. 9, 2004, which is incorporated herein by reference in its entirety.

### BACKGROUND

A wide variety of medical conditions and disorders have been successfully treated using miniature implantable medical devices. For example, one type of implantable medical device is an implantable stimulator. Implantable stimulators stimulate internal tissue, such as nerves, by emitting an electrical stimulation current according to programmed stimulation parameters.

One class of implantable stimulators, also known as BION® devices (where BION® is a registered trademark of Advanced Bionics Corporation, of Valencia, Calif.), are typically characterized by a small housing containing electronic circuitry that produces an electric stimulation current between spaced electrodes. These stimulators, also referred to as microstimulators, are implanted proximate to the target tissue so that the stimulation current produced by the electrodes stimulates the target tissue to reduce symptoms or otherwise provide therapy for a wide variety of conditions and disorders.

For example, urinary urge incontinence may be treated by stimulating the nerve fibers proximal to the pudendal nerves of the pelvic floor. Erectile or other sexual dysfunctions may be treated by providing stimulation of the cavernous nerve(s). Other disorders, e.g., neurological disorders caused by injury or stroke, may be treated by providing stimulation to other appropriate nerve(s).

An example of an implantable device for tissue stimulation is described in U.S. Pat. No. 5,312,439, “Implantable Device Having an Electrolytic Storage Electrode.” U.S. Pat. No. 5,312,439 is incorporated herein by reference in its entirety.

Another exemplary microstimulator is described in U.S. Pat. No. 5,193,539, “Implantable Microstimulator,” which patent is also incorporated herein by reference in its entirety. This patent describes a microstimulator in which power and information for operating the microstimulator are received through a modulated, alternating magnetic field. This is accomplished with a coil in the microstimulator that is adapted to function as the secondary winding of a transformer. This induction coil receives energy from an external device outside the patient's body. A capacitor is then used to store the received electrical energy. This stored energy can be used to generate a stimulation current through the microstimulator's exposed electrodes under the control of electronic control circuitry.

In U.S. Pat. Nos. 5,193,540 and 5,405,367, which patents are incorporated herein by reference in their respective entireties, a structure and method of manufacture for an implantable microstimulator are disclosed. The microstimulator has a structure which is manufactured to be substantially encapsulated within a hermetically-sealed housing that is inert to body fluids. The microstimulator structure is also of a size and shape capable of implantation in a living body with appropriate surgical tools. Within the microstimulator, an induction coil receives energy or data from outside the patient's body.

In yet another example, U.S. Pat. No. 6,185,452, which patent is likewise incorporated herein by reference in its entirety, discloses a device configured for implantation beneath a patient's skin for the purpose of nerve or muscle stimulation and/or parameter monitoring and/or data communication. Such a device contains a power source for powering the internal electronic circuitry. This power supply is a battery that may be externally charged each day. Similar battery specifications are found in U.S. Pat. No. 6,315,721, which patent is additionally incorporated herein by reference in its entirety.

In another example, such microstimulator systems prevent and/or treat various disorders associated with prolonged inactivity, confinement or immobilization of one or more muscles. Such microstimulators are taught, for example, in U.S. Pat. No. 6,061,596 “Method for Conditioning Pelvis Musculature Using an Implanted Microstimulator;” U.S. Pat. No. 6,051,017, “Implantable Microstimulator and Systems Employing the Same;” U.S. Pat. No. 6,175,764, “Implantable Microstimulator System for Producing Repeatable Patterns of Electrical Stimulation;” U.S. Pat. No. 6,181,965, “Implantable Microstimulator System for Prevention of Disorders;” U.S. Pat. No. 6,185,455, “Methods of Reducing the Incidence of Medical Complications Using Implantable Microstimulators;” and U.S. Pat. No. 6,214,032, “System for Implanting a Microstimulator.” These patents are incorporated herein by reference in their respective entireties.

Implantable medical devices, such as a stimulator, are often intended to permanently remain within the body of a patient. Hence, transcutaneous communication between an implanted medical device and an external device is often important for the implanted medical device to continue functioning properly over its useful life. For example, communication with an implanted medical device may be effected to perform a number of functions including, but not limited to, transferring power to the implanted device, transferring data to and from the implanted device, programming the implanted device, and monitoring the implanted device's various functions.

This transcutaneous communication between an implanted medical device and an external device is often facilitated by the use of coils that are configured to emit and/or receive magnetic fields. For example, the external device may include a primary coil configured to emit and/or receive a magnetic field that is used to communicate with and/or transfer power to an implanted medical device. Likewise, the implanted medical device may include a secondary coil configured to emit and/or receive a magnetic field that is used to communicate with and/or receive power from the external device.

### SUMMARY

Systems for communicating with or transferring power to an implantable medical device include a primary coil configured to emit a magnetic field and a secondary coil in the implantable medical device configured to receive the magnetic field. The primary coil includes at least one butterfly coil.

Methods of communicating with or transferring power to an implantable medical device include emitting a magnetic field with a primary coil and receiving the magnetic field with a secondary coil. The primary coil includes at least one butterfly coil.

### BRIEF DESCRIPTION OF THE DRAWINGS

The accompanying drawings illustrate various embodiments of the present invention and are a part of the specification. The illustrated embodiments are merely examples of the present invention and do not limit the scope of the invention.

FIG. 1 shows an exemplary implantable medical device and an exemplary external device according to principles described herein.

FIG. 2 shows a number of exemplary external devices that may be used to communicate with and/or transfer power to the implantable medical device according to principles described herein.

FIG. 3 is a functional block diagram of an exemplary external device according to principles described herein.

FIG. 4 shows a conventional coil that may be used as a primary coil in an external device and/or as a secondary coil in an implantable medical device.

FIG. 5 illustrates a typical configuration wherein the primary coil of an external device is located at or near the outer surface of the patient's skin and the secondary coil of an implantable medical device is located at or near the inner surface of the patient's skin.

FIG. 6A is a top view of an exemplary butterfly coil that may be used as the primary coil of an external device and/or the secondary coil of an implantable medical device according to principles described herein.

FIG. 6B shows the magnetic field induced by the butterfly coil of FIG. 6A according to principles described herein.

FIGS. 7A-7E illustrate a number of exemplary butterfly coils having different wing shapes according to principles described herein.

FIG. 8 illustrates an exemplary configuration wherein a butterfly coil is used as the primary coil of an external device and a conventional single loop coil is used as the secondary coil of an implantable medical device according to principles described herein.

FIG. 9A shows a top view of an exemplary primary coil and an exemplary secondary coil that are both butterfly coils according to principles described herein.

FIG. 9B shows the primary and secondary coils orthogonally oriented according to principles described herein.

FIG. 10 illustrates a dual butterfly coil configuration according to principles described herein.

FIG. 11 illustrates a third butterfly coil that is aligned with one of the butterfly coils in the dual butterfly coil configuration of FIG. 10 according to principles described herein.

FIG. 12 shows a butterfly coil that has been constructed using a single continuous wire according to principles described herein.

FIG. 13 illustrates an exemplary wing of a butterfly coil with stacked turns according to principles described herein.

FIG. 14 illustrates an exemplary wing of a butterfly coil with turns that have been wound one around another in the same plane according to principles described herein.

Throughout the drawings, identical reference numbers designate similar, but not necessarily identical, elements.

### DETAILED DESCRIPTION

Systems and methods of communicating with or transferring power to an implantable medical device are described herein. A primary coil is configured to emit a magnetic field, and a secondary coil in the implantable medical device is configured to detect or receive the magnetic field. The primary coil and/or secondary coils may include one or more butterfly coils. The butterfly coils are configured to optimize coupling between the primary and secondary coils.

In the following description, for purposes of explanation, numerous specific details are set forth in order to provide a thorough understanding of the present systems and methods. It will be apparent, however, to one skilled in the art that the present systems and methods may be practiced without these specific details. Reference in the specification to “one embodiment” or “an embodiment” means that a particular feature, structure, or characteristic described in connection with the embodiment is included in at least one embodiment. The appearance of the phrase “in one embodiment” in various places in the specification are not necessarily all referring to the same embodiment.

The terms “implantable medical device” and “implanted medical device” will be used interchangeably herein and in the appended claims to refer to any medical device or component that can be implanted within a patient and that is configured to transcutaneously communicate with and/or receive power from an external device. The implantable medical device may include, but is not limited to, a stimulator, a microstimulator, an implantable pulse generator (IPG) coupled to one or more leads having a number of electrodes, a spinal cord stimulator (SCS), a cochlear implant, a deep brain stimulator, a drug pump, a micro-drug pump, a pacemaker, a defibrillator, a functional electrical stimulation (FES) system, a blood pump, an implantable sensor, or any combination of these or other medical devices or components that are implanted within the patient.

Exemplary stimulators and microstimulators suitable for use as described herein include, but are not limited to, those disclosed in U.S. Pat. Nos. 5,193,539; 5,193,540; 5,312,439; 6,185,452; 6,164,284; 6,208,894; and 6,051,017. Exemplary IPGs suitable for use as described herein include, but are not limited to, those disclosed in U.S. Pat. Nos. 6,381,496, 6,553,263; and 6,760,626. Exemplary spinal cord stimulators suitable for use as described herein include, but are not limited to, those disclosed in U.S. Pat. Nos. 5,501,703; 6,487,446; and 6,516,227. Exemplary cochlear implants suitable for use as described herein include, but are not limited to, those disclosed in U.S. Pat. Nos. 6,219,580; 6,272,382; and 6,308,101. Exemplary deep brain stimulators suitable for use as described herein include, but are not limited to, those disclosed in U.S. Pat. Nos. 5,938,688; 6,016,449; and 6,539,263. Exemplary drug pumps suitable for use as described herein include, but are not limited to, those disclosed in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,360,019; 4,487,603; 4,562,75; 4,627,850; 4,678,408; 4,685,903; 4,692,147; 4,725,852; 4,865,845; 5,057,318; 5,059,423; 5,080,653; 5,097,122; 5,112,614; 5,137,727; 5,234,692; 5,234,693; 5,728,396; 6,368,315; 6,740,072; and 6,770,067. Exemplary micro-drug pumps suitable for use as described herein include, but are not limited to, those disclosed in U.S. Patent Publication No. 2004/0082908 and U.S. Pat. Nos. 5,234,692; 5,234,693; 5,728,396; 6,368,315; 6,666,845; and 6,620,151. All of these listed patents and publications are incorporated herein by reference in their respective entireties.

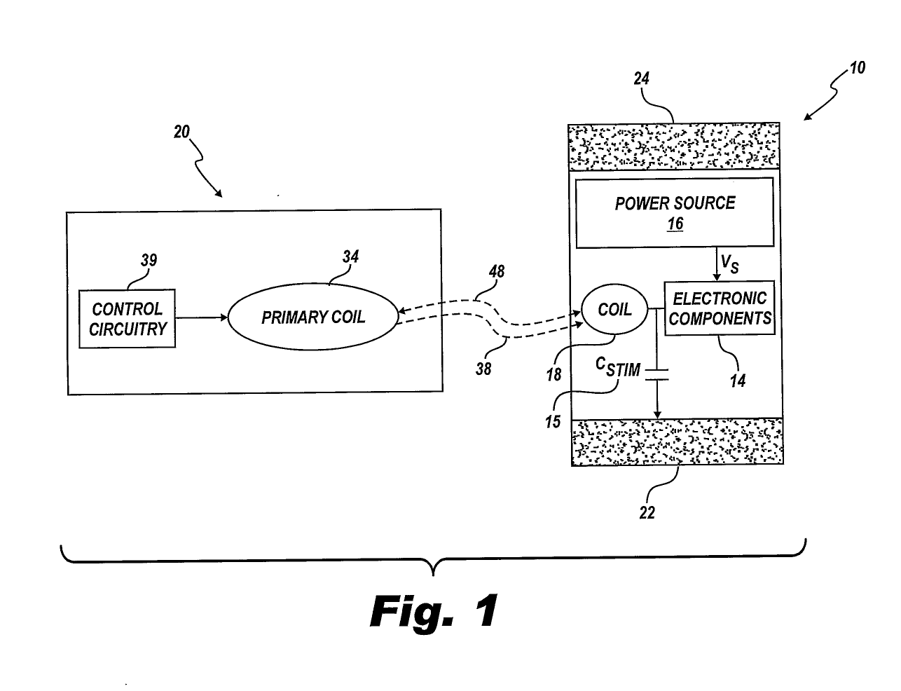

By way of example, an exemplary implantable medical device will be described in connection with FIG. 1. FIG. 1 illustrates an exemplary implantable microstimulator (10). The implantable microstimulator (10) is merely illustrative of the many different implantable medical devices that may be used in connection with the methods and systems described herein and should not be considered as limiting in any way.

FIG. 1 shows an exemplary implantable microstimulator (10) and an exemplary external device (20). The microstimulator (10) may be configured to stimulate tissue to treat any number of conditions, diseases, or disorders. For example, the microstimulator (10) may be used to reduce pain, promote normal tissue function, prevent atrophy, or otherwise provide therapy for various disorders. The stimulation applied by the microstimulator (10) may include electrical stimulation, drug stimulation, chemical stimulation, thermal stimulation, electromagnetic stimulation, mechanical stimulation, and/or any other suitable stimulation.

The implantable microstimulator (10) may be implanted within a patient using any suitable implantation technique and the external device (20) may be used to communicate with and/or transfer power to the microstimulator (10). Such communication and/or power transfer may include, but is not limited to, transcutaneously transmitting data to the microstimulator (10), receiving data from the microstimulator (10), transferring power to a power source (16) in the microstimulator (10), and/or providing recovery power to the power source (16) when the battery is in a battery depletion state. As used herein and in the appended claims, unless otherwise specifically denoted, the term “battery depletion state” will be used to refer to a state wherein the power source (16) has been depleted to a voltage level substantially equal to zero volts.

As illustrated in FIG. 1, the implantable microstimulator (10) may include a number of components. The power source (16) is configured to output a voltage Vs used to supply the various components within the microstimulator (10) with power and/or to generate the power used for electrical stimulation. The power source (16) may be a primary battery, a rechargeable battery, super capacitor, a nuclear battery, a mechanical resonator, an infrared collector (receiving, e.g., infrared energy through the skin), a thermally-powered energy source (where, e.g., memory-shaped alloys exposed to a minimal temperature difference generate power), a flexural powered energy source (where a flexible section subject to flexural forces is part of the stimulator), a bioenergy power source (where a chemical reaction provides an energy source), a fuel cell, a bioelectrical cell (where two or more electrodes use tissue-generated potentials and currents to capture energy and convert it to useable power), an osmotic pressure pump (where mechanical energy is generated due to fluid ingress), or the like. Alternatively, the microstimulator (10) may include one or more components configured to receive power from another medical device that is implanted within the patient.

In instances where the power source (16) is a battery, it may be a lithium-ion battery or other suitable type of battery. If the power source (16) is a rechargeable battery, it may be recharged by the external device (20) through a power link such as a radio frequency (RF) power link. One type of rechargeable battery that may be used is described in International Publication WO 01/82398 A1, published Nov. 1, 2001, and/or WO 03/005465 A1, published Jan. 16, 2003, both of which are incorporated herein by reference in their entireties. Other battery construction techniques that may be used to make the power source (16) include those shown, for example, in U.S. Pat. Nos. 6,280,873; 6,458,171, and U.S. Patent Publication Nos. 2001/0046625 A1 and 2001/0053476 A1, all of which are incorporated herein by reference in their respective entireties.

The microstimulator (10) may also include a coil (18), referred to herein and in the appended claims, unless otherwise specifically denoted, as a secondary coil. The secondary coil (18) is configured to receive and/or emit a magnetic field that is used to communicate with and/or receive power from the external device (20) and/or another implantable medical device. Such communication and/or power transfer may include, but is not limited to, transcutaneously receiving data from the external device (20), transmitting data to the external device (20), and/or receiving power used to recharge the power source (16).

In some embodiments, the microstimulator (10) may include a stimulating capacitor (15) and two or more leadless electrodes (22, 24) configured to stimulate tissue within a patient with electric current. The electrodes (22, 24) may be made of a conducting ceramic, conducting polymer, stainless steel, and/or a noble or refractory metal, such as gold, silver, platinum, iridium, tantalum, titanium, titanium nitride, niobium or their alloys. One of the electrodes (e.g., 24) may be designated as a stimulating electrode to be placed close to the stimulation site and one of the electrodes (e.g., 22) may be designated as an indifferent electrode used to complete a stimulation circuit.

Either or both of the electrodes (22, 24) may alternatively be located at the ends of short, flexible leads as described in U.S. patent application Ser. No. 09/624,130, filed Jul. 24, 2000, which is incorporated herein by reference in its entirety. The use of such leads permits, among other things, electrical stimulation to be directed more locally to targeted tissue(s) a short distance from the surgical fixation of the bulk of the microstimulator (10), while allowing most elements of the microstimulator (10) to be located in a more surgically convenient site. This minimizes the distance traversed and the surgical planes crossed by the microstimulator (10).

The external surfaces of the microstimulator (10) may be composed of biocompatible materials. For example, the external surface of the microstimulator (10) may be made of glass, ceramic, polymers, metal, or any other material that provides a hermetic package that will exclude water vapor but permit passage of magnetic fields used to transmit data and/or power.

The microstimulator (10) may be implanted within a patient with a surgical tool such as a hypodermic needle, bore needle, or any other tool specially designed for the purpose. Alternatively, the microstimulator (10) may be implanted using endoscopic or laparoscopic techniques.

The exemplary external device (20) of FIG. 1 may include control circuitry (39) and a coil (34) configured to emit and/or receive a magnetic field that is used to communicate with and/or transfer power to the microstimulator (10). The coil (34) will be referred to herein and in the appended claims, unless otherwise specifically denoted, as a primary coil. In some examples, the primary coil (34) and the secondary coil (18) of the microstimulator (10) communicate by sending RF signals across a bidirectional telemetry link (48). The RF signals sent across the bidirectional telemetry link (48) may be modulated using frequency shift keying (FSK) or by some other modulation scheme. The primary coil (34) and the coil (18) of the microstimulator (10) may also communicate via a unidirectional telemetry link (3 8). The unidirectional telemetry link (38) may use an on/off keying (OOK) modulation scheme. The unidirectional telemetry link (38) is also known as an OOK telemetry link. On/off keying (OOK) modulation is frequency independent and is also known as pulse width modulation (PWM).

The external device (20) may be configured to perform any number of functions via the bidirectional telemetry link (48) and/or the unidirectional telemetry link (38). As mentioned, the external device (20) may be configured to transcutaneously charge the rechargeable power source (16) in the implanted microstimulator (10), transcutaneously transmit data to the microstimulator (10), and/or transcutaneously receive data from the microstimulator (10) via the bidirectional telemetry link (48) and/or the unidirectional telemetry link (3 8). The transmitted data may include stimulation parameters, configuration bits, programming bits, calibration bits, and/or other types of data.

The functions performed by the external device (20) will vary as best serves the particular application of the microstimulator (10). The shape and design of the external device (20) will likewise vary. For example, the external device (20) may include a chair pad and a base station. In use, the chair pad may be placed on a chair and a patient who has an implanted microstimulator (10) may sit on the chair pad to recharge the power source (16) in the microstimulator (10) and/or to transfer data between the base station and the microstimulator (10). Alternatively, the external device (20) may be housed within a casing that is worn by the patient near the surface of the skin. In general, the external device (20) may be any device configured to communicate with and/or transfer power to an implantable microstimulator (10).

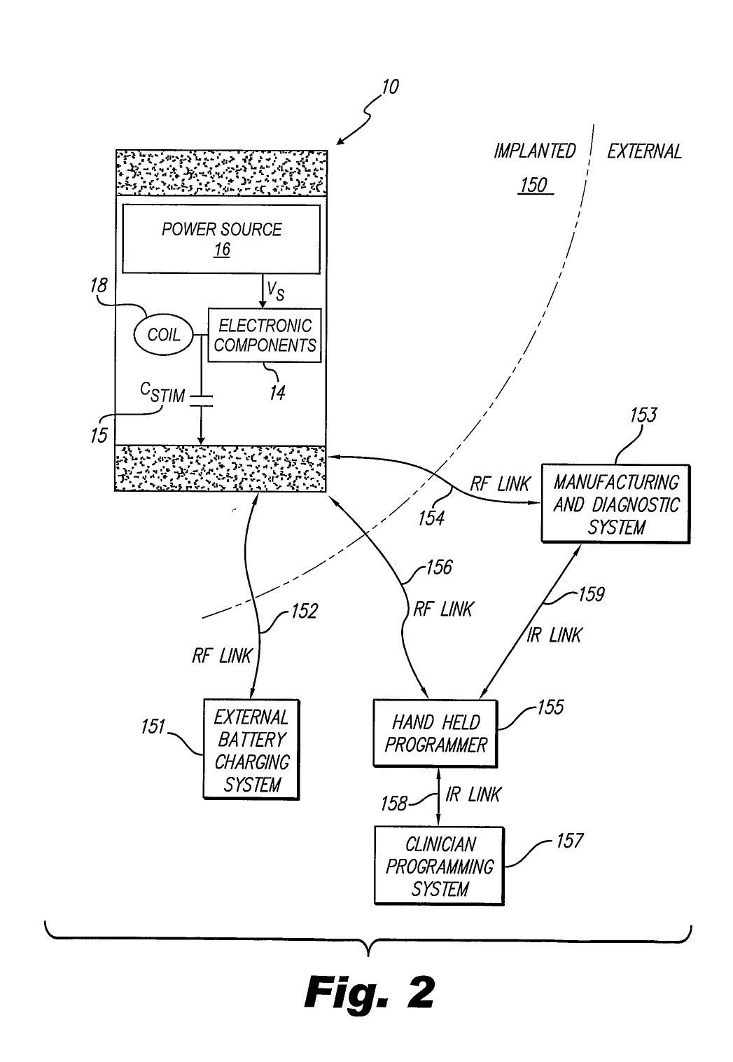

In some embodiments, as shown in FIG. 2, multiple external devices may be used to communicate with and/or transfer power to the microstimulator (10). For example, an external battery charging system (EBCS) (151) may provide power used to recharge the power source (16) via an RF link (152). External devices including, but not limited to, a hand held programmer (HHP) (155), clinician programming system (CPS) (157), and/or a manufacturing and diagnostic system (MDS) (153) may be configured to activate, deactivate, program, and test the microstimulator (10) via one or more RF links (154, 156). It will be recognized that the RF links (152, 154, 156) may be any type of link such as an optical link, a thermal link, or any other energy-coupling link.

Additionally, if multiple external devices are used in the treatment of a patient, there may be some communication among those external devices, as well as with the implanted microstimulator (10). For example, the CPS (157) may communicate with the HHP (155) via an infrared (IR) link (158), with the MDS (153) via an IR link (161), and/or directly with the microstimulator (10) via an RF link (160). These communication links (158, 161, 160) are not limited to IR and RF links and may include any other type of communication link. Likewise, the MDS (153) may communicate with the HHP (155) via an IR link (159) or via any other suitable communication link.

The HHP (155), MDS (153), CPS (157), and EBCS (151) are merely illustrative of the many different external devices that may be used in connection with the microstimulator (10). Furthermore, it will be recognized that the function performed by any two or more of the HHP (155), MDS (153), CPS (157), and EBCS (151) maybe performed by the single external device (20) of FIG. 1. One or more of the external devices (153, 155, 157) maybe embedded in a seat cushion, mattress cover, pillow, garment, belt, strap, pouch, or the like so as to be positioned near the implanted microstimulator (10) when in use.

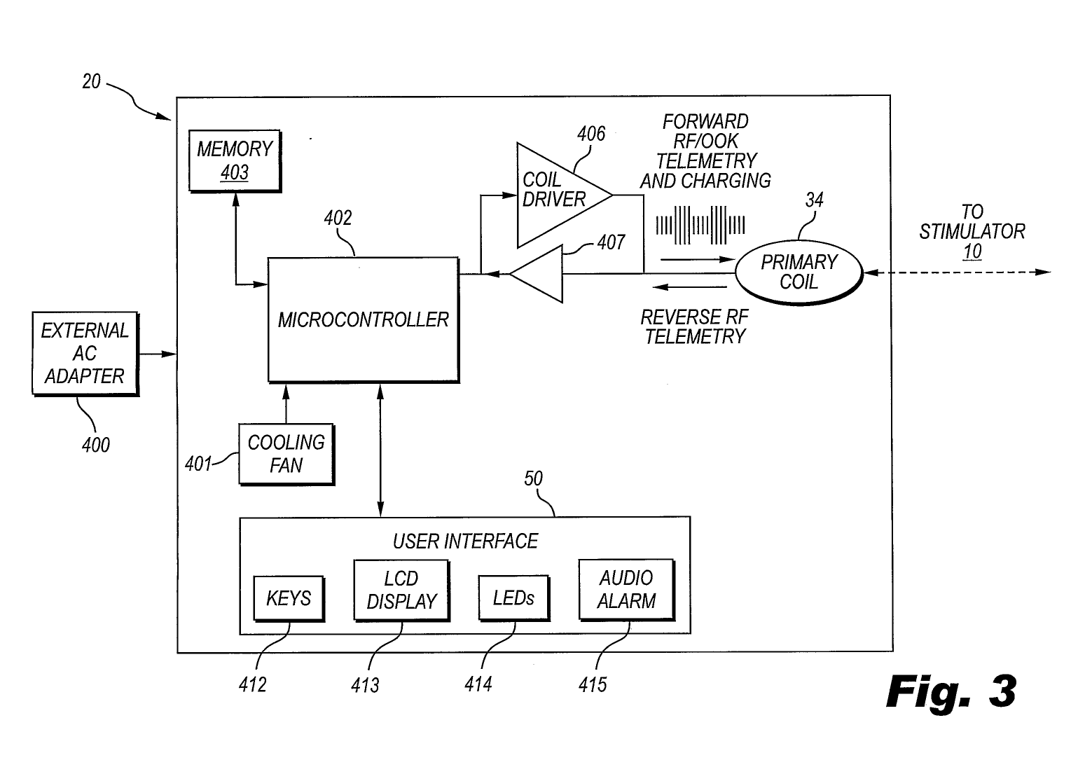

FIG. 3 is a functional block diagram of an exemplary external device (20) according to principles described herein. As shown in FIG. 3, the external device (20) may include a number of components, some or all of which are configured to facilitate the transfer of power and/or data to and from the implantable stimulator (10). For example, the illustrated external device (20) may include memory (403), the primary coil (34), a coil driver circuit (406), a user interface (50), and a microcontroller (402). The microcontroller (402) is configured to control the operation of the various components included in the external device (20). A cooling fan (401) may be included to cool the microcontroller (402). The external device (20) may be powered, for example, by an external alternating current (AC) adapter (400). Alternatively, the external device (20) may be powered by a battery or by some other power source.

As shown in FIG. 3, the user interface (50) may include user input keys (412), one or more LCD displays (413), one or more LED displays (414) and/or an audio alarm (415). These controls may assist a user in controlling the external device (20) and/or the stimulator (10). For example, the audio alarm (415) may be used to indicate to the user when the external device (20) has finished charging the stimulator's power source (16; FIG. 1). The audio alarm (415) may also be used as a signal indicator for any other system event or mode.

The external device (20) may further include a receiver (407) configured to receive reverse telemetry signals from the implantable stimulator (10). The receiver (407) may be an amplifier or any other component configured to receive telemetry signals. These signals may then be processed by the microcontroller (402).

As mentioned above, primary and secondary coils (34, 18) in the external device (20) and the implanted device, respectively, allow for communication and/or power transfer between the external device (20) and the implanted medical device such as the stimulator (10) described in connection with FIG. 2. As used herein and in the appended claims, unless otherwise specifically denoted, the term “primary coil” will refer to any coil that is a part of an external device, and the term “secondary coil” will refer to any coil that is a part of an implantable medical device. It will be recognized that communication and/or power transfer may occur between a primary coil and one or more secondary coils, between two or more secondary coils, and/or between two or more primary coils. The examples given herein describe a communication and/or power transfer scheme between a primary coil of an external device (20) and an implanted medical device for illustrative purposes only. However, it will be recognized that the communication and/or power transfer may be between one or more implantable medical devices or between one or more external devices (20).

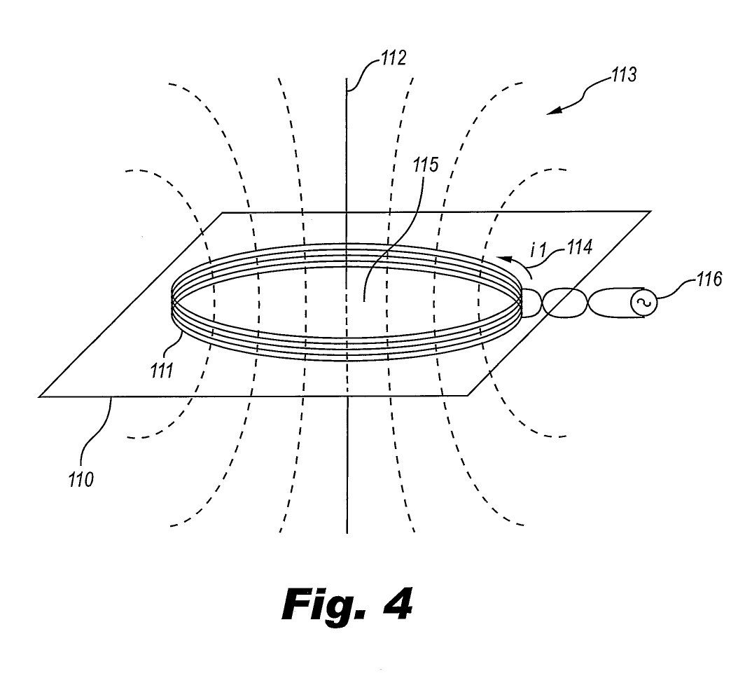

In some external devices and implantable medical devices, the primary and secondary coils are in the shape of a circular loop. FIG. 4 shows a conventional coil (111) that may be used as the primary coil (34; FIG. 1) and/or secondary coil (18; FIG. 1). As shown in FIG. 4, the coil (111) is in the shape of a substantially circular loop and includes one or more turns of conductive wire. It will be recognized that the substantially circular shape of the coil (111) is merely illustrative. As used herein and in the appended claims, unless otherwise specifically denoted, the term “turn” refers to a complete loop of wire in a coil. For example, the coil (111) of FIG. 4 includes five turns of wire. However, it will be recognized that the coil (111) may include any number of turns. The turns of the coil (111) define a center opening or aperture (115) having a central axis (112).

FIG. 4 shows that the coil (111) is typically placed on or near the outer surface of the skin (110) when used as a primary coil (34; FIG. 1) to communicate with an implanted medical device having a secondary coil (18; FIG. 1). Hence, as shown in FIG. 4, the coil (111) lays on or along the skin (110) such that the central axis (112) of the coil (111) is perpendicular to the surface of the skin (110). It will be recognized that the surface of the skin (110) is not always flat. Hence, the central axis (112) of the coil (111) is sometimes only approximately or substantially perpendicular to the surface of the skin (110).

FIG. 4 also depicts a number of magnetic flux lines (113) that are generated when a time-varying current i₁ (114) circulates in the coil (111). Time-varying current i₁ (114) is generated by a current source (116) that is electrically coupled to the coil (111). The time-varying current i₁ (114) circulating in the coil (111) causes the coil (111) to behave like a magnetic dipole and generate a corresponding magnetic field represented by the magnetic flux lines (113). As the current i₁ (114) varies with time, the magnetic flux lines (113) vary with time as well. As shown in FIG. 4, where the lines of magnetic flux (113) pass through the surface of the skin (110), the lines of magnetic flux (113) are essentially perpendicular to the surface of the skin (110). In addition, the magnetic flux lines (113) near the center of the aperture (115) are substantially parallel to the central axis (112) of the coil (111).

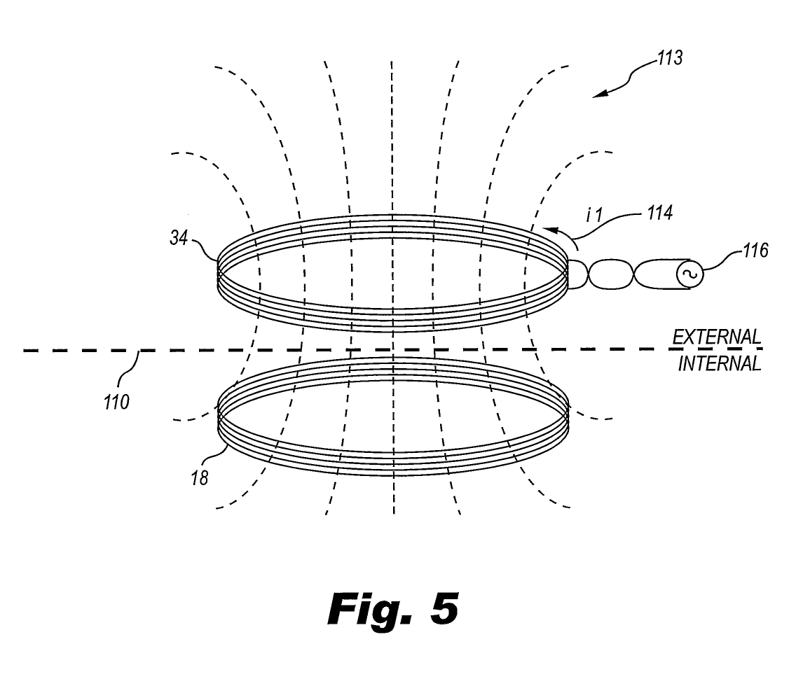

FIG. 5 illustrates a typical configuration in which an external device's primary coil (34) is located at or near the outer surface of the patient's skin (110) and an implantable medical device's secondary coil (18) is located at or near the inner surface of the patient's skin (110). As shown in FIG. 5, the primary coil (34) generates a number of magnetic flux lines (113) which pass through the surface of the skin (110). The secondary coil (18) has to be coaxially aligned with the primary coil (18) to a certain degree so that the secondary coil (18) can detect, receive, or be affected by, the emitted magnetic flux lines (113). The emitted magnetic flux lines (113) generated by the primary coil (34) cause a current to flow in the aligned secondary coil (18). The induced magnetic field component generated by the primary coil (34) that has the most effect on the secondary coil (18) is the component perpendicular to the skin (110).

However, a number of implantable medical devices are implanted deep within a patient and/or may be oriented in any direction with respect to the surface of the skin. For example, a microstimulator may be implanted next to tissue that is deep within the patient and oriented so as to optimize stimulation of that target tissue. In these instances, the secondary coil (18) contained within the implanted medical device may be aligned in any direction with respect to the surface of the skin. For example, the central axis of the secondary coil (18) may be substantially parallel with the surface of the skin as opposed to being substantially perpendicular as illustrated in FIG. 5. Hence, it is often desirable to increase the parallel component of the induced magnetic field generated by the primary and/or secondary coils in order to optimize communication and/or power transfer between the coils without unduly increasing the amount of current required to drive the coils.

This can be accomplished using a butterfly coil design for either or both of the primary and secondary coils (34, 18; FIG. 1). As will be described in more detail below, an exemplary butterfly coil design includes two wings, or coils, in which current flows in opposite directions. In some examples, the wings are coplanar. Alternatively, the wings may be bent as best serves a particular application. Exemplary butterfly coil designs that may be used as the primary and/or secondary coils (34, 18; FIG. 1), according to principles disclosed herein, will now be described. In some examples, the butterfly coil designs described herein are configured to increase the induced magnetic field component parallel to the skin, as compared to a conventional circular loop coil of the same inductance carrying the same amount of current. The ability to provide stronger magnetic fields with less inductance allows butterfly coils to be driven at higher frequencies than conventional single loop coils. Furthermore, various characteristics of butterfly coils may be adjusted to optimize communication and/or power transfer between an external device and an implanted medical device for different implanted medical device locations and orientations.

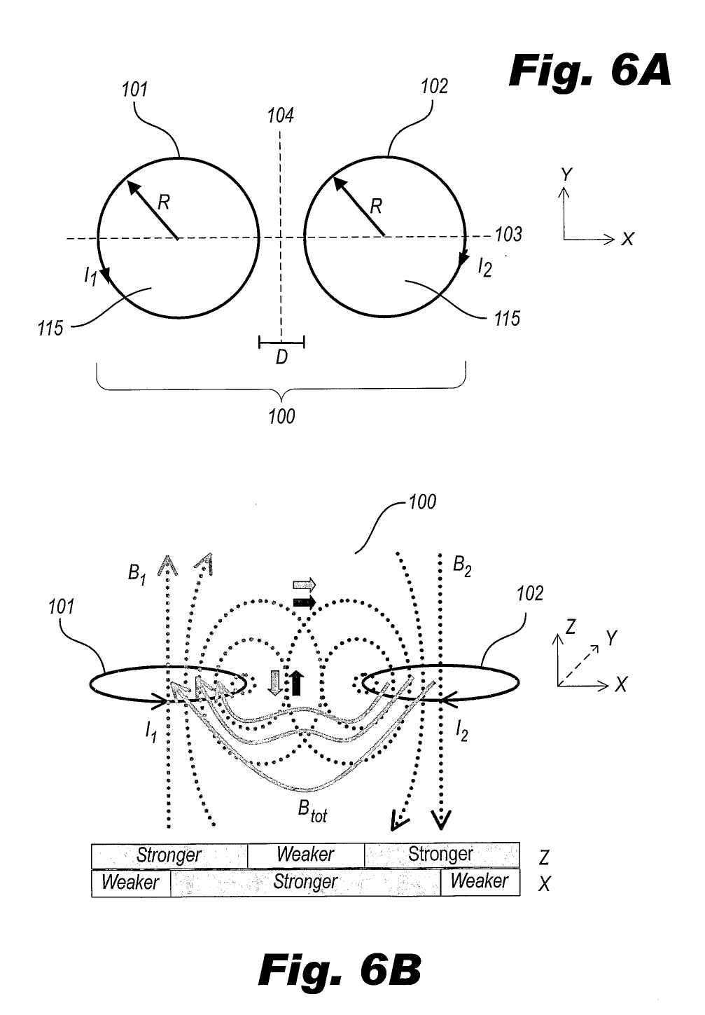

FIG. 6A is a top view of an exemplary butterfly coil (100) that may be used as the primary coil (34; FIG. 1) of an external device (20; FIG. 1) and/or the secondary coil (18; FIG. 1) of an implantable medical device (10; FIG. 1). As shown in FIG. 6A, the butterfly coil (100) includes at least a first wing (101) and a second wing (102). The first and second wings (101, 102) are coils that are each in the shape of a circular loop with radii R, much like the circular loop shown in FIG. 4. In some embodiments, the butterfly coil (100) includes more than two wings. As will be described in more detail below, the first and second wings (101, 102) may have any shape and/or size.

As shown in FIG. 6A, the first wing (101) and the second wing (102) are substantially mirror images of each other. It will be recognized, however, that the first wing (101) may have a different shape and/or size than the second wing (102) as best serves a particular application.

The first and second wings (101, 102) of the butterfly coil (100) may include any number of turns of conductive wires. For example, each wing (101, 102) may include five turns as illustrated in connection with FIG. 4. In some embodiments, each wing (101, 102) only includes one turn of conductive wire. The wire may be made out of any conductive or semi-conductive material (e.g., copper) as best serves a particular application.

As shown in FIG. 6A, each wing (101, 102) is centered along a first axis (103) which corresponds to the coordinate X. The first axis (103) is shown to extend in the horizontal (X) direction for illustrative purposes only and may be oriented in any other direction as best serves a particular application. The two wings (101, 102) are separated by a distance D and are equidistant from a second axis (104). The separation distance D may be any distance as best serves a particular application. The second axis (104), corresponding to the coordinate Y, is shown to extend in the vertical (Y) direction for illustrative purposes only and may be oriented in any other direction as best serves a particular application.

Each wing (101, 102) of the butterfly coil (100) includes a center opening or aperture (115). Each aperture (115) maybe hollow (i.e., an air core). Alternatively, each aperture (115) may be at least partially filled with a ferrite or other suitable material (i.e., a ferrite core). In yet another alternative example, a portion of a toroid, a curved ferromagnetic, or a curved material having a suitable magnetic permeability may serve as the core for both wings (101, 102) to increase the magnetic flux between the wings (101, 102).

As shown in FIG. 6A, the butterfly coil (100) is constructed such that current flows in opposite directions in each wing (101, 102). For example, a first current I₁ flows in a counter-clockwise direction in the first wing (101) and a second current I₂ flows in a clockwise direction in the second wing (102). The first and second currents I₁ and I₂, respectively, may be equal in magnitude and phase. Alternatively, the two currents may have different magnitudes and/or phases. As will be described in more detail below, the opposite flow of current in each of the wings (101, 102) induces a magnetic field that has a stronger component parallel to the skin than that produced by the traditional single loop coil (111) of FIG. 4.

The wings (101, 102) of the butterfly coil (100) may be completely separated one from another, as shown in FIG. 6A. When the wings (101, 102) are separated one from another, the first wing (101) is coupled to a first current source and the second wing (102) is coupled to a second current source. The first and second current sources are configured to cause I₁ and I₂ to flow in opposite directions.

Alternatively, as will be described in more detail below, the wings (101, 102) may be electrically coupled. For example, a single wire may be used to construct the entire butterfly coil (100). Alternatively, a conducting wire may be coupled to the first and second wings (101, 102) to electrically couple the wings (101, 102).

FIG. 6B shows the magnetic field Bₜₒₜ induced by the butterfly coil (100). The butterfly coil is shown to be parallel to the X-Y plane for illustrative purposes. In other words, the first axis (103; FIG. 6A) corresponds to the X coordinate and the second axis (104; FIG. 6A) corresponds to the Y coordinate. The magnetic field lines induced by the first and second wings (101, 102) of the butterfly coil (100) are in the Z and X directions.

As shown in FIG. 6B, the first wing (101) induces a first magnetic field B₁ and the second wing (102) induces a second magnetic field B₂. Because the current I₁ in the first wing (101) flows in a counter clockwise direction, the first magnetic field B₁ exits the first wing (101) in the positive Z direction. However, as shown in FIG. 6B, the current I₂ in the second wing (102) flows in a clockwise direction. Therefore, the second magnetic field B₂ exits the second wing (102) in the negative Z direction.

The induced magnetic fields B₁ and B₂ are added to yield the total induced magnetic field Bₜₒₜ of the butterfly coil (100). The table located at the bottom of FIG. 6B compares the magnitude of Bₜₒₜ with the magnetic field of the conventional circular loop coil (111) of FIG. 4 for different regions of the butterfly coil (100). It is assumed for comparative purposes that the conventional circular loop coil (111) of FIG. 4 is located in the same position and has the same size as the first wing (101).

For example, in the region between the two wings (101, 102) (shown as distance D in FIG. 6A), the Z component of Bₜₒₜ is weaker than the Z component of the magnetic field of the conventional circular loop coil (111; FIG. 4) because the magnetic fields B₁ and B₂ are oriented in opposite directions. Hence, when added together, the Z components of B₁ and B₂ within this middle region effectively cancel each other out. As mentioned previously, the Z component of Bₜₒₜ is perpendicular to the skin. Thus, the perpendicular component of the magnetic field Bₜₒₜ is effectively decreased in the region between the two wings (101, 102) by the butterfly coil configuration (100).

However, as shown in FIG. 6B, the X components of B₁ and B₂ are oriented in the same direction in the region between the center axes of the two wings (101, 102). Therefore, when B₁ and B₂ are added together in this region, the X component of Bₜₒₜ in any plane parallel with the X-Y plane is stronger than the X component of the magnetic field of the conventional circular loop coil (111; FIG. 4). As mentioned previously, the X component of Bₜₒₜ is parallel to the skin. Thus, the parallel component of the magnetic field Bₜₒₜ is effectively increased in between the center axes of the two wings (101, 102) by the butterfly coil configuration (100).

The table of FIG. 6B also shows that the Z component of Bₜₒₜ is stronger than the Z component of the magnetic field induced by the conventional circular loop coil (111; FIG. 4) in the regions enclosed by each wing (101, 102). This is because the Z components of B₁ and B₂ are oriented in the same direction within these regions. On the other hand, the X component of Bₜₒₜ is weaker than the X component of the magnetic field of the conventional circular loop coil (111; FIG. 4) in between the center axes and distal edges of each of the wings (101, 102), respectively.

In many applications, it is often desired to induce a large magnetic field with a coil having a small inductance. Coils with larger inductances are more difficult to operate at higher frequencies, have lower self-resonant frequencies and larger impedances, require higher voltages and are more difficult to tune. Hence, it is often desired to maximize the fraction of magnetic field strength to coil inductance (B/L) at a given distance from the coil. The butterfly coil design (100) of FIG. 6A represents an optimal case of B/L because the mutual inductance between the first and second wings (101, 102) represent a negligible amount (usually less than ten percent) of the total inductance of the butterfly coil (100). Assuming that this mutual inductance is equal to zero, it can be shown that the conventional circular loop coil (111; FIG. 4) requires approximately 1.4 times more turns than the butterfly coil (100) of FIG. 6A to generate the same magnetic field. Hence, if both coils have the same inductance and current levels, the butterfly coil (100) can generate a magnetic field up to 1.4 times larger than the magnetic field generated by the conventional circular loop coil (111; FIG. 4). Furthermore, the butterfly coil (100) of FIG. 6A dissipates heat more efficiently than does the conventional circular loop coil (111) of FIG. 4.

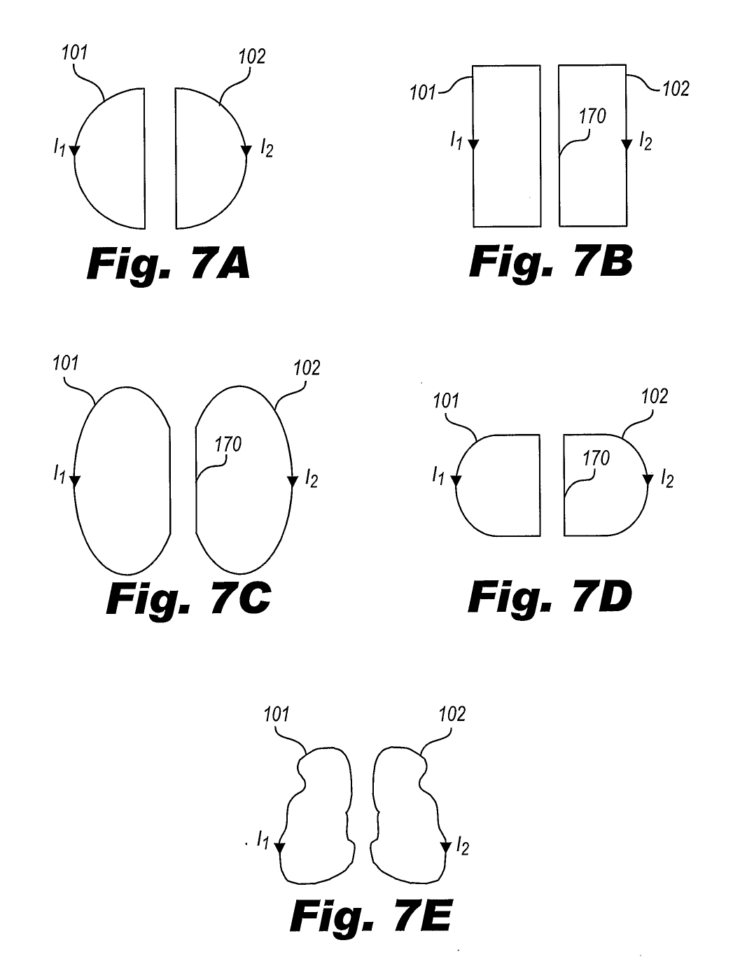

As mentioned, the wings (101, 102) of the butterfly coil (100) may have any shape. For example, FIGS. 7A-7E illustrate a number of exemplary butterfly coils having different wing shapes. It will be recognized that the shapes of the butterfly coils shown in FIGS. 7A-7E are merely illustrative of the many different shapes that may be used in connection with the methods and systems described herein. For example, the wings (101, 102) of the butterfly coil (100) maybe in the shape of half circles (FIG. 7A), rectangles (FIG. 7B), partial ellipses (FIG. 7C), the letter “D” (FIG. 7D), or any other arbitrary shape (FIG. 7E).

In some examples, as shown in FIGS. 7B-7D, a portion of each wing (101, 102) most proximal to the axis separating the two wings (101, 102) may include a substantially straight segment (170) which is parallel to the coil axis. These straight segments (170) are configured to provide a more homogenous distribution of the electromagnetic fields in particularly the components oriented in the X direction, along the Y axis.

In some embodiments, the circumference, shape, orientation, number of turns, and/or separation distance D of the wings (101, 102) may be adjusted to account for any orientation and/or any implantation depth of the implantable medical device (10; FIG. 1). The angle between the wings (101, 102) may additionally or alternatively be adjusted to account for any orientation and/or any implantation depth of the implantable medical device (10; FIG. 1). Furthermore, the angle of the primary coil (10; FIG. 1) with respect to the surface of the skin may be adjusted to account for any orientation of the implantable medical device (10; FIG. 1).

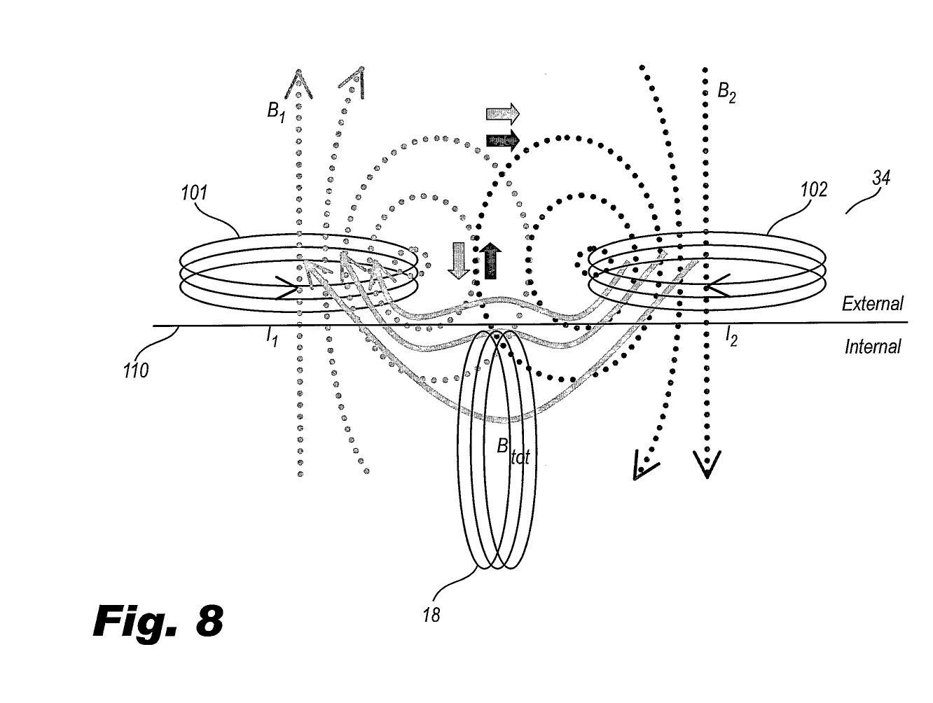

FIG. 8 illustrates an exemplary configuration wherein a butterfly coil is used as an external device's primary coil (34) and a conventional single loop coil is used as an implantable medical device's secondary coil (18). As shown in FIG. 8, the central axis of the secondary coil (18) is substantially parallel with the surface of the patient's skin (110). In this orientation, the secondary coil (18) is configured to detect or be affected by magnetic fields that are also parallel with the surface of the skin (110). Hence, the use of the butterfly coil as the primary coil (34) is advantageous in many communication and/or power transfer configurations because the butterfly coil is configured to optimize the induced magnetic field component parallel to the skin (110) in the region in between the two wings (101, 102). It will be recognized that the secondary coil (18) may alternatively include a butterfly coil configured to communicate with and/or receive power from a conventional single loop coil.

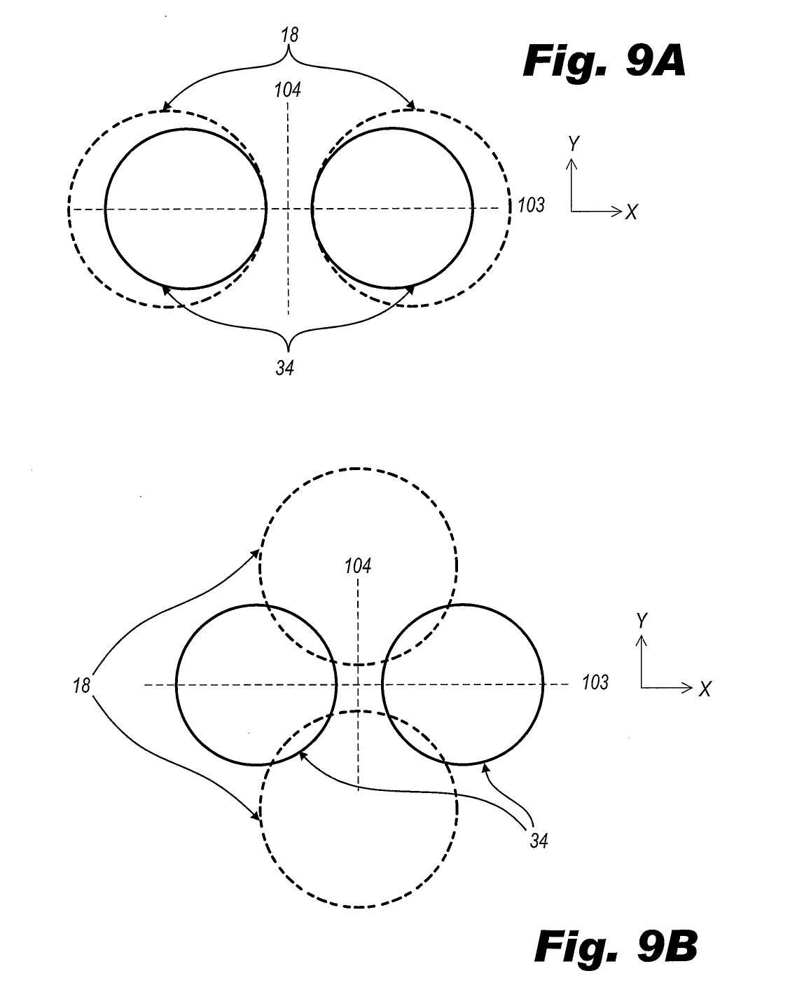

In some alternative examples, a butterfly coil is used as both the primary and secondary coils (34, 18; FIG. 4). For example, FIG. 9A shows a top view of an exemplary primary coil (34) and an exemplary secondary coil (18) that are both butterfly coils. The wings of the secondary coil (18) are illustrated using dashed lines and are larger in circumference for illustrative purposes only. It will be recognized that the shape and size of the wings of both the primary coil (34) and the secondary coil (18) may be modified as best serves a particular application.

As shown in FIG. 9A, the primary coil (34) may be aligned over the secondary coil (18) such that the wings of the primary coil (34) substantially cover the wings of the secondary coil (18). In other words, the primary and secondary coils (18) have the same X-Y plane orientation. This orientation optimizes coupling between the primary and secondary coils (34, 18) and therefore may be used to optimize communication and/or power transfer between the coils (34, 18).

Alternatively, as shown in FIG. 9B, the primary and secondary coils (34, 18) may be orthogonally oriented such that there is minimal coupling between the coils (34, 18). For example, as shown in FIG. 9B, the primary coil (34) is oriented along the first axis (103) and the secondary coil (18) is oriented along the second axis (104). In this orientation, the magnetic field generated by one coil (e.g., the primary coil (34)) is substantially rejected by the other coil (e.g., the secondary coil (18)).

Hence, the orientation of the primary and/or secondary coils (34, 18) may be adjusted to filter out undesirable signals or magnetic fields. For example, the implantable device (10; FIG. 1) may include two secondary coils that are orthogonally positioned-one for receiving communication data from the external device (20; FIG. 1) and the other for receiving power from the external device (20; FIG. 1). Hence, when the primary coil (34) is aligned with the first secondary coil, it may only send communication data to the implantable device (10; FIG. 1). Likewise, when the primary coil (34) is aligned with the second secondary coil, it may only transfer power to the implantable device (10; FIG. 1).

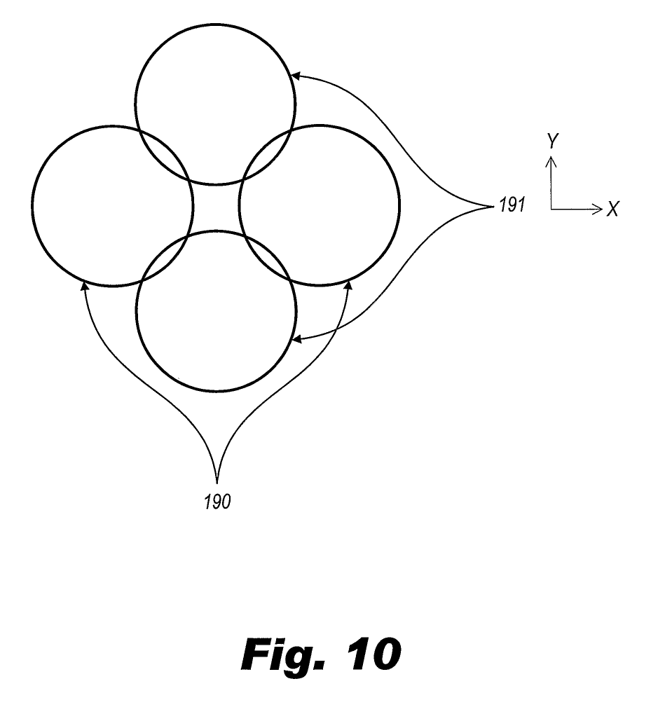

In some embodiments, as shown in FIG. 10, the primary and/or secondary coils (34, 18) may include a dual butterfly coil configuration. A dual butterfly coil configuration, such as that shown in FIG. 10, includes two butterfly coils (190, 191) orthogonally oriented in the same plane. As shown in FIG. 10, the first butterfly coil (190) and the second butterfly coil (191) are located in the same X-Y plane and are orthogonal to each other.

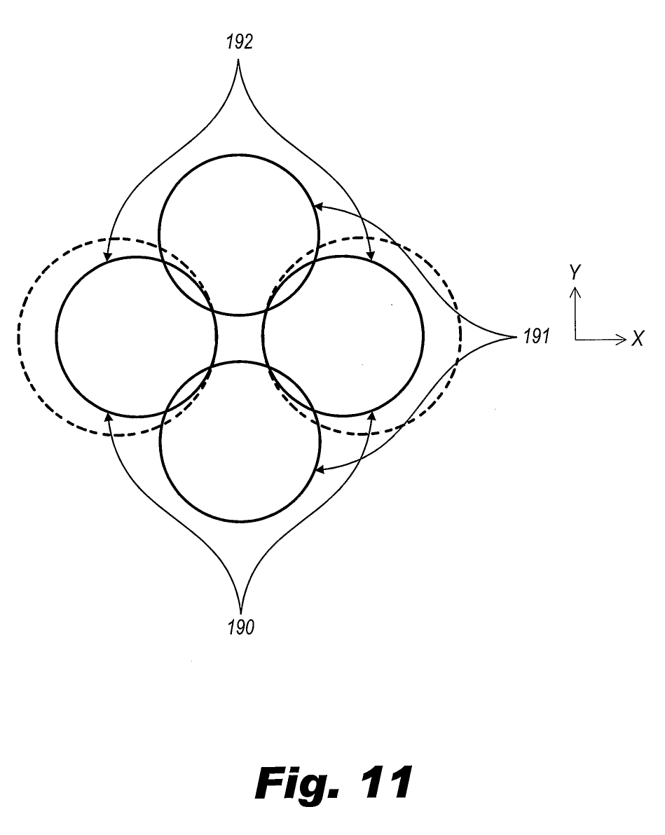

The exemplary dual butterfly coil configuration of FIG. 10 may be used as a primary coil (34; FIG. 1) in an external device (20; FIG. 1) and/or as a secondary coil (18; FIG. 1) in an implantable medical device (10; FIG. 1) and may be used to filter out undesirable signals or magnetic fields, as described in connection with FIG. 9B. For example, as shown in FIG. 11, the first and second butterfly coils (190, 191) may be included in the implantable medical device (10; FIG. 1) as the secondary coil (18; FIG. 1) and a third butterfly coil (192) may be included in the external device (20; FIG. 1) as the primary coil (34; FIG. 1). If the third butterfly coil (192) is aligned with the first butterfly coil (190), as shown in FIG. 11, the third butterfly coil (192) may communicate with and/or transfer power to the implantable medical device (10; FIG. 1) via the first butterfly coil (190) without interacting with the second butterfly coil (191). Likewise, if the third butterfly coil (192) is aligned with the second butterfly coil (191), the third butterfly coil (192) may communicate with and/or transfer power to implantable medical device (10; FIG. 1) via the second butterfly coil (191) without interacting with the first butterfly coil (190).

It will be recognized that the first and second butterfly coils (190, 191) may alternatively be included in the external device (20; FIG. 1) as the primary coil (34; FIG. 1) and that the third butterfly coil (192) may alternatively be included in the implantable medical device (10; FIG. 1) as the secondary coil (18; FIG. 1). In yet other alternative embodiments, the external device (20; FIG. 1) and the implantable medical device (10; FIG. 1) each include dual butterfly coil configurations.

Exemplary methods of constructing a butterfly coil (100; FIG. 6A) will now be described. It will be recognized that the methods described herein may be modified as best serves a particular application. As mentioned, the first and second wings (101, 102) of the butterfly coil (100) may include any number of turns of conductive wires. The wire may be made out of any conductive or semi-conductive material (e.g., copper) as best serves a particular application.

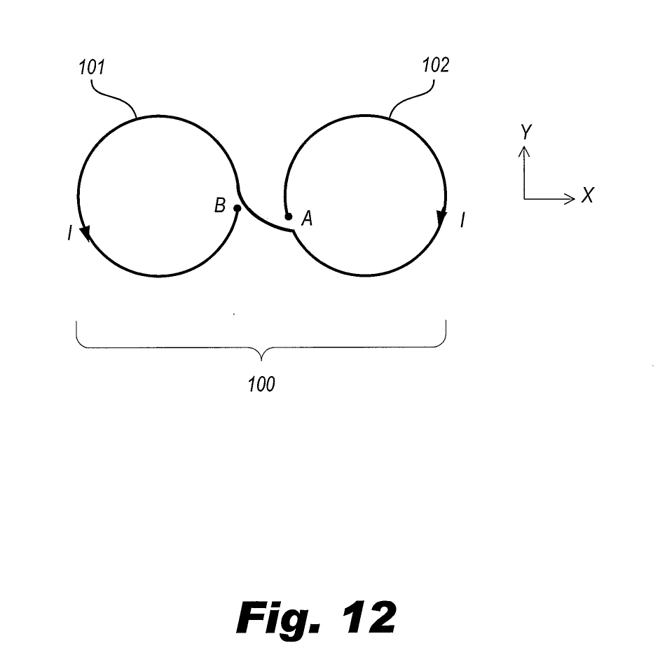

In some embodiments, each wing (101, 102) of the butterfly coil (100) is wound or constructed independently and then connected in series to allow for the correct opposite current flow. Alternatively, as shown in FIG. 12, a single continuous wire is used to construct both wings (101, 102) of the butterfly coil (100). The continuous wire is wound such that when a current source is connected to the ends (labeled “A” and “B”) of the continuous wire, current flows in opposite directions in the two wings (101, 102). The continuous wire may be monofilar or it may be composed of many strands of wore (e.g., Litz wire.)

Where each wing (101, 102) of the butterfly coil (100) of FIG. 12 includes multiple turns, one wing (e.g., 102) may be completely wound before the second wing (e.g., 101) is wound. Alternatively, the wings (101, 102) may be constructed by winding successive full turns. A full turn includes one turn for each wing (101, 102).

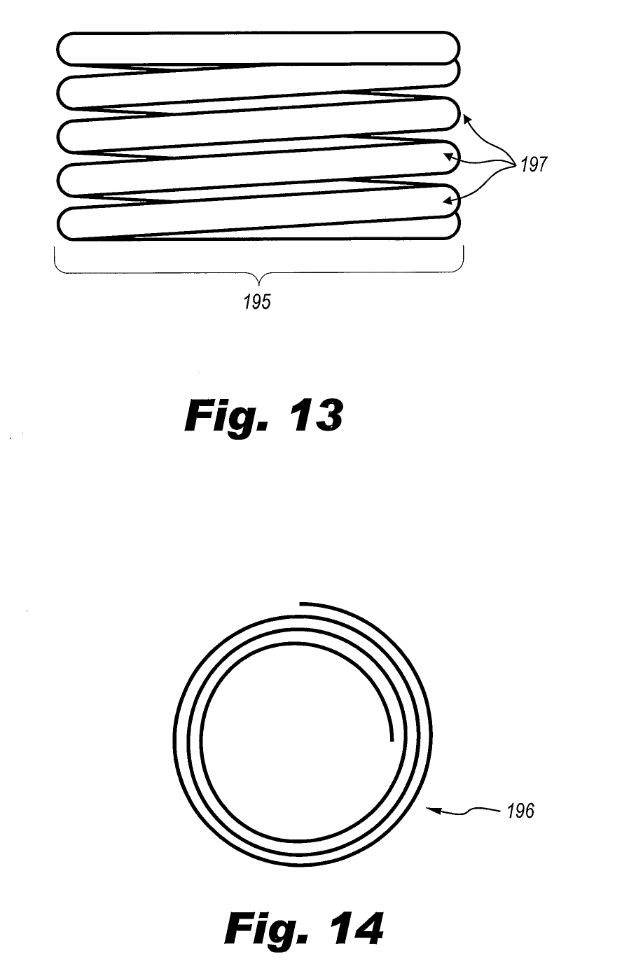

The spread and positioning of the wire used in the butterfly coil (100) may also be adjusted as best serves a particular application. For example, the turns of the butterfly coil (100) may be stacked one on top of another. FIG. 13 illustrates an exemplary wing (195) of a butterfly coil (100; FIG. 6A) with stacked turns (197). Alternatively, as shown in FIG. 14, a flat or “pancake” technique may be used to construct the butterfly coil (100; FIG. 6A). FIG. 14 illustrates an exemplary wing (196) of a butterfly coil (100; FIG. 6A) with turns that have been wound one around another in the same plane. The flat or “pancake” technique may be used when it is desirable for the butterfly coil to have a low profile. In some embodiments, a combination of flat and stacking techniques may be used in constructing the butterfly coil (100; FIG. 6A).

The preceding description has been presented only to illustrate and describe embodiments of the invention. It is not intended to be exhaustive or to limit the invention to any precise form disclosed. Many modifications and variations are possible in light of the above teaching.

What is claimed is:

1. A system for communicating with or transferring power to an implantable medical device, said system comprising:
a primary coil configured to emit a magnetic field, said primary coil comprising a first butterfly coil; and
a secondary coil in said implantable medical device configured to receive said magnetic field.

2. The system of claim 1, wherein said first butterfly coil comprises:
a first wing having one or more turns of wire; and
a second wing having one or more turns of wire;
wherein said first and second wings are coplanar and are separated by a separation distance.

3. The system of claim 2, wherein said first and second wings have a shape comprising at least one or more of a circle, a half-circle, a rectangle, a partial ellipse, and a letter “D”.

4. The system of claim 2, wherein said one or more turns of wire in said first and second wings are positioned in a single plane.

5. The system of claim 2, wherein said turns of wire in said first and second wings are stacked one on top of another.

6. The system of claim 2, wherein said first and second wings are constructed with a single continuous wire.

7. The system of claim 2, wherein said first and second wings are constructed using separate wires.

8. The system of claim 1, wherein said primary coil further comprises a second butterfly coil orthogonal to said first butterfly coil, wherein said first and second butterfly coils are located in a common plane.

9. The system of claim 1, wherein said butterfly coil comprises at least one or more of an air core, a ferrite core, a portion of a toroid core, and a curved material.

10. The system of claim 1, wherein said secondary coil comprises a second butterfly coil.

11. The system of claim 1, wherein said secondary coil is configured to emit a second magnetic field and said primary coil is configured to receive said second magnetic field.

12. The system of claim 1, wherein:
said primary coil is configured emit a magnetic field having a component parallel to said skin;
wherein said parallel component of said magnetic field is received by said secondary coil when said secondary coil is oriented within a patient such that a central axis of said secondary coil is substantially parallel to the skin of said patient.

13. A device configured to communicate with or transfer power to an implantable medical device, said device comprising:
a primary coil configured to emit a magnetic field carrying communication data or power for said implantable medical device;
wherein said primary coil comprises a first butterfly coil.

14. The device of claim 13, wherein said first butterfly coil comprises:
a first wing having one or more turns of wire; and
a second wing having one or more turns of wire;
wherein said first and second wings are coplanar and are separated by a separation distance.

15. The device of claim 14, wherein said first and second wings have a shape comprising at least one or more of a circle, a half-circle, a rectangle, a partial ellipse, and a letter “D”.

16. The device of claim 14, wherein said one or more turns of wire in said first and second wings are positioned in a single plane.

17. The device of claim 14, wherein said turns of wire in said first and second wings are stacked one on top of another.

18. The device of claim 14, wherein said first and second wings are constructed with a single continuous of wire.

19. The device of claim 14, wherein said first and second wings are constructed using separate wires.

20. The device of claim 13, wherein said primary coil further comprises a second butterfly coil orthogonal to said first butterfly coil, wherein said first and second butterfly coils are located in a common plane.

21. The device of claim 13, wherein said butterfly coil comprises at least one or more of an air core, a ferrite core, a portion of a toroid core, and a curved material.

22. The device of claim 13, wherein said implantable medical comprises a secondary coil oriented such that a central axis of said secondary coil is substantially parallel to the skin of a patient.

23. An implantable medical device configured to communicate with or receive power from an external device, said implantable medical device comprising:
a secondary coil configured to emit or receive a magnetic field carrying communication data or power for said implantable medical device;
wherein said secondary coil comprises a first butterfly coil.

24. The medical device of claim 23, wherein said first butterfly coil comprises:
a first wing having one or more turns of wire; and
a second wing having one or more turns of wire;
wherein said first and second wings are coplanar and are separated by a separation distance.

25. The medical device of claim 24, wherein said first and second wings have a shape comprising at least one or more of a circle, a half-circle, a rectangle, a partial ellipse, and a letter “D”.

26. The medical device of claim 24, wherein said one or more turns of wire in said first and second wings are positioned in a single plane.

27. The medical device of claim 24, wherein said turns of wire in said first and second wings are stacked one on top of another.

28. The medical device of claim 24, wherein said first and second wings are constructed with a single continuous wire.

29. The medical device of claim 24, wherein said first and second wings are constructed using separate wires.

30. The medical device of claim 23, wherein said secondary coil further comprises a second butterfly coil orthogonal to said first butterfly coil, wherein said first and second butterfly coils are located in a common plane.

31. The medical device of claim 23, wherein said butterfly coil comprises at least one or more of an air core, a ferrite core, a portion of a toroid core, and a curved material.

32. A method of communicating with or transferring power to an implantable medical device, said method comprising:
emitting a magnetic field with a primary coil, said primary coil comprising a first butterfly coil; and
receiving said magnetic field with a secondary coil in said implantable medical device.

33. The method of claim 32, wherein said first butterfly coil comprises:
a first wing having one or more turns of wire; and
a second wing having one or more turns of wire;
wherein said first and second wings are coplanar and are separated by a separation distance.

34. The method of claim 33, further comprising modifying at least one or more of said turns of wire in said first and second wings, said separation distance, a shape of said first and second wings, and a size of said first and second wings to optimize coupling between said primary and secondary coils.

35. The method of claim 33, further comprising positioning said one or more turns of wire in said first and second wings in a single plane.

36. The method of claim 33, further comprising stacking said turns of wire in said first and second wings one on top of another.

37. The method of claim 33, further comprising using a single continuous wire to construct said first and second wings.

38. The method of claim 33, further comprising using separate wires to construct said first and second wings.

39. The method of claim 32, wherein said primary coil further comprises a second butterfly coil orthogonal to said first butterfly coil, wherein said first and second butterfly coils are located in a same plane.
